(19) United States
(12) Patent Application Publication
(10) Pub. No.: US 2005/0042711 A1
(43) Pub. Date: Feb. 24, 2005

(54) Field test for fungi

(75) Inventor: Jenefir Isbister, Potomac, MD (US)

Correspondence Address: BROWDY AND NEIMARK, P.L.L.C.;624 NINTH STREET, NW, SUITE 300, WASHINGTON, DC, 20001-5303, US

(73) Assignee: GEORGE MASON UNIVERSITY, Fairfax, VA (US)

(21) Appl. No.: 10/637,542

(22) Filed: Aug. 11, 2003

(51) Int. Cl. C12Q 1/18; C12Q 1/04

(52) U.S. Cl. 435/32; 435/34

(57) ABSTRACT

A method for detecting fungi in a liquid matrix which also contains bacteria comprising adding an effective amount of at least one compound which inhibits a pathway by which bacteria synthesize lysine to inhibit bacterial growth and allowing for growth of the fungi for subsequent detection. Compounds for this method include nicotinic acid, or analogues of dihydropicolinic acid, and analogues of diaminopimelic acid.

### FIELD TEST FOR FUNGI

1. Field of the Invention

The present invention relates to an assay for fungal contaminants.

2. Background of the Invention

Exposure to fungi may have significant health implications. Fungi have now been found to cause conditions such as chronic sinusitis, asthma, and allergies. In addition, exposure to fungi has been correlated with conditions such as sick-building syndrome, infantile pulmonary hemorrhage, neurological disorders, and other related conditions. However, there has been no way to correlate the symptoms exhibited with exposure to fungi.

In particular, asthma rates in the United States and in many other parts of the world have nearly doubled. The number of asthma sufferers is now more than 17 million in the United States alone, with an estimated five million of them children. The death rate for children from asthma increased by 78% between 1980 and 1993. The cost of medical care for asthma, including hospitalization and treatment, exceeds $14 billion a year.

Sick building syndrome (SBS) is a complex condition that may involve many factors. However, the occurrence of fungi in buildings affected with SBS has been positively correlated with the condition.

The most common chronic disease in the United States is sinusitis. It afflicts more than 37 million Americans. This sinus condition is characterized by inflammation of the membranes of the nose and sinus cavity. Symptoms can include runny nose, nasal congestion, headaches, and polyp growth in the sinus cavities. Recently, the cause of this disease was found to be fungi.

In order to determine if one has been exposed, or is currently being exposed, to certain fungi, the presence of the fungi must be documented in samples or areas of exposure. Unfortunately, in many samples, bacteria are present along with the fungi, and it is difficult to isolate fungi for detection from liquid matrices in which mixed microbial populations may be present. As noted above, many health conditions can be caused by exposure to fungi, and it is important to identify the cause of the condition so that appropriate treatment can be commenced.

In many samples taken to analyze for the presence of fungi, there are also many bacteria. Under less than optimal growth conditions for fungi, bacteria grow more rapidly, resulting in limitation of nutrient availability for the fungi. This results in an overgrowth of bacteria, which masks the fungal growth. In general, a defined medium such as a base medium for a fungal assay does not provide optimal nutrients for rapid growth of fungi, even through the pH and temperature of incubation are selected for optimal fungal growth.

### SUMMARY OF THE INVENTION

It is an object of the present invention to overcome the aforesaid deficiencies in the prior art.

It is another object of the present invention to grow true fungi without bacterial contamination.

It is yet another aspect of the present invention to assay for fungi without interference from bacteria present in the sample.

The present invention provides a medium for growth of true fungi while inhibiting bacterial growth so as to facilitate the detection and isolation of fungi from samples such as liquid matrices in which mixed microbial populations may be present. Bacterial growth is inhibited by including in the medium a compound which inhibits bacterial growth by blocking the pathway for bacterial synthesis of an essential amino acid not provided in the medium.

The medium of the present invention inhibits growth of bacteria, but does not affect the growth of true fungi. This medium makes it possible to detect low levels of fungi in the presence of bacteria in a sample. The medium can be used to detect low or high levels of fungal contamination in liquid matrices where fungal contamination is undesirable, i.e., medical solutions, environmental samples, tissue culture media, or buffer solutions. The medium supports only the growth of true fungi while preventing growth of other microbes that have a similar growth habit.

Nicotinic acid, an analogue of dihydropicolinic acid, is an inhibitor of bacterial lysine synthesis. Analogues of diamino-pimelic acid, and compounds with similar structures, are also expected to be competitive inhibitors of the pathway by which bacteria synthesize lysine, an essential amino acid. Including at least one of these inhibitors in a medium for microorganism growth thus permits growth of fungi but inhibits growth of bacteria, making it possible to isolate fungi for assay. Compounds can readily be tested to determine if the compound is a competitive inhibitor for bacterial growth by adding the compound to a medium for culturing bacteria which does not contain lysine. If the bacteria fails to grow, the compound is an inhibitor.

The inhibitors can be added to any defined growth medium that does not contain lysine. The defined medium used as a base for fungal assay will support growth of both bacteria (prokaryotes) and fungi (eukaryotes) in the absence of nicotinic acid. Bacteria and fungi use different pathways for synthesizing the essential amino acid lysine. The inhibitors inhibit bacterial synthesis of lysine, while allowing the fungi to synthesize lysine via a different pathway. One skilled in the art can readily determine which analogues of dihydropicolinic acid and diaminopimelic acid are inhibitors of bacterial synthesis of lysine, and therefore determine which analogues will be useful in the present invention.

### DETAILED DESCRIPTION OF THE INVENTION

Addition of at least one inhibitor of bacterial synthesis of lysine to a medium on which both bacteria and fungi grow, which medium does not contain lysine, makes it possible to grow the fungi while inhibiting the growth of the bacteria. While the bacteria are not killed by the inhibitor, their growth is inhibited such that it is possible to culture the fungi in a sample and to recover true fungi without substantial bacterial contamination. Among these inhibitors are nicotinic acid, analogues of nicotinic acid, analogues of dihydropicolinic acid, and analogues of diaminopimelic acid.

The medium on which both bacteria and fungi grow generally includes a carbon source, a nitrogen source, suitable vitamins, and inorganic substances. For the purposes of the present invention, no lysine is present in the medium. The carbon source can be derived from at least one of the following: starch, glucose, monosaccharides, polysaccharides, dextrin, maltose, saccharose, methyl cellulose, fructose, furanose, and corn powder. The nitrogen source can be derived from one of the following: defatted soybean powder, peptone, yeast paste, yeast syrup, peanut cake powder, yeast powder, wheat bran, casein, calcium caseinate, and defatted beancake powder.

The following non-limiting examples illustrate the method of the present invention.

ISTOR medium was developed for isolating fungi from aqueous environments containing a mixed population of bacteria and fungi. A master mix of ISTOR medium was prepared from ingredients listed in Table I. Aliquots of the Master Mix were weighed out and placed into sterile containers. The appropriate volume of sterile distilled water was added to each container to create ISTOR medium to mimic ampoule mixing. As shown, 239 mg of Master Mix was required for each ampoule to accommodate 8 mL of aqueous sample. The pH of the ISTOR medium when reconstituted was about 5.5 (when autoclaved, the pH is a bit lower).

Microorganisms, fungi and bacteria, were inoculated into ISTOR medium and incubated at room temperature. Observations were made over an extended nine day incubation period. As shown in Table II, all strains of fungi inoculated into ISTOR grew well. None of the bacteria tested demonstrated growth over this extended incubation period.

TABLE I
MASTERMIX PREPARATION

| | 1 L (G) | 100 mL (G) | 8 mL (G) |
|---|---|---|---|
| KH2PO4 | 0.72 | 0.072 | 0.006 |
| K2HPO4 | 16.5 | 1.650 | 0.132 |
| Glucose | 10.00 | 1.00 | 0.080 |
| MgSO4 | 0.50 | 0.050 | 0.004 |
| NaNO3 | 0.87 | 0.087 | 0.007 |
| Yeast Ex | 0.30 | 0.030 | 0.003 |
| Nic. Ac. | 12.30 | 1.23 | 0.098 |
| TOTAL (G) | 41.19 | 4.19 | 0.329 |

TABLE II
FUNGI/BACTERIA MAXTERMIX TRIAL

| FUNGI | GROWTH | BACTERIA | GROWTH |
|---|---|---|---|
| Jeniculosporium | + | S. pneumoniae | − |
| Aeremonium | + | E. coli | − |
| Cylindrocarpon | + | B. subtilis | − |
| Illosporium | + | P. aeruginosa | − |
| Fusarium coccophilum | + | | |

Fungal cultures of the fungi listed in Table III were obtained from Dr. A. Torzilli, the mycologist at George Mason University on the Manassas campus. Each fungus was cultured on Potato Dextrose Agar for use in these studies.

Bacteria tested for growth in ISTOR are listed in Table IV. Each bacterium was cultured in liquid medium for inoculation into ISTOR for growth.

TABLE III
FUNGAL STRAINS EVALUATED

| Strains | From | Medium | T° |
|---|---|---|---|
| Jeniculosporium | Dr. Torzilli | PDA | RT |
| Aeremonium | Dr. Torzilli | PDA | RT |
| Cylindrocarpon | Dr. Torzilli | PDA | RT |
| Illosporium | Dr. Torzilli | PDA | RT |
| Fusarium coccophilum | Dr. Torzilli | PDA | RT |

TABLE IV
BACTERIA EVALUATED FOR GROWTH IN ISTOR

| Strains | From | Medium | T° |
|---|---|---|---|
| S. pneumoniae (Gram +) | ATCC | BHIA | 35 |
| E. coli (Gram −) | ATCC | TSA | 35 |
| B. subtilis (Gram +) | ATCC | TSA | 30 |
| P. aeruginosa (Gram −) | ATCC | TSA | 35 |

The base ISTOR medium has the following composition:

| | |
|---|---|
| Glucose | 1% |
| MgSO4 | 0.05% |
| NaNO3 | 0.087% |
| Yeast Extract | 0.03% |

The medium was prepared in potassium phosphate buffer at pH 5.7. The pH was readjusted with potassium hydroxide after nicotinic acid addition to pH 5.0.

Fungal and bacterial cultures were inoculated into base medium with varying concentrations of nicotinic acid, as shown in Table V. With nicotinic acid present at 0.1 M, none of the bacteria inoculated into the medium grew, whereas all of the fungi tested exhibited growth over a total observation period of three to four days.

TABLE V
EVALUATION OF NICOTINIC ACID CONCENTRATIONS

| Strain/(Nic. Ac. M) | 0 | 0.002 | 0.01 | 0.02 | 0.05 | 0.1 |
|---|---|---|---|---|---|---|
| Jeniclosporium | + | | | | | + |
| Aeremonium | | | | | | + |
| Cylindrocarpon | + | + | + | + | + | + |
| Illosporium | | | | | | + |
| Fusarium coccophilum | | | | | | + |
| S. pneumoniae | + | +/− | − | − | − | − |
| E. coli | + | | | +/− | +/− | − |
| B. subtilis | | | | | | − |
| P. aeruginosa | | | | | | − |

Experiments were conducted to study the effect of the pH of the ISTOR medium on the growth of fungi and bacteria. A medium was prepared from the following:

| | |
|---|---|
| Glucose | 1% |
| MgSO4 | 0.05% |
| NaNO3 | 0.087% |
| Yeast Extract | 0.03% |
| Nicotinic acid | 1.23% (0.1M) |

The medium was prepared in potassium phosphate buffer at pH 7.5 and then divided into different tubes. The tubes were brought to different pH values with HCl prior to being autoclaved. Table VI indicates the medium at each of the pH values tested:

TABLE VI
ISTOR @ DIFFERENT pH - DESCRIPTION

| pH Value | Description |
|---|---|
| 7.6 | Very Dark |
| 6.5 | Yellowish |
| 5.5 | Clear/Colorless |
| 4.5 | Precipitation of NA begins |

Microorganisms used for these tests and the pH of each medium tested are given in Table VII. It can be seen from Table VII that the medium at pH values near pH 6 provided the best growth of the fungi. Bacterial growth was not observed in the medium at pH 6.5 to pH 4.5. A slight bacterial growth for S. pneumoniae was observed at pH 7.5. However, this pH is not optimal for growth of fungi.

TABLE VII
MICROORGANISMS TESTED WITH ISTOR
AT VARIOUS PH VALUES

| Strains/pH | 7.6 | 6.5 | 5.5 | 4.5 |
|---|---|---|---|---|
| Jeniculosporium | +++ | ++ | ++ | − |
| Aeremonium | ++ | ++ | ++ | − |
| Cylindrocarpon | +++ | +++ | +++ | − |
| Illosporium | − | ++ | +++ | − |
| Fusarium coccophilum | − | ++ | ++ | − |
| S. pneumoniae | +/− | − | − | − |
| E. coli | − | − | − | − |

Because nicotinic acid is not very soluble in water, provision of nicotinic acid via a pad containing the appropriate quantity of nicotinic acid for 8 mL samples was not feasible.

The environmental mud collected on the George Mason University campus was extracted for microorganisms. Different dilutions of the mud were plated on TSA (bacteria) and PDA (fungi) to determine which organisms were present. Thirteen bacterial colonies were isolated, and seven fungi were isolated from the environmental sample. Each bacterial isolate was inoculated onto Trypticase Soy Agar, and each fungal isolate was inoculated onto Potato Dextrose Agar to serve as stocks for testing with ISTOR. The data are shown in Table VIII. None of the thirteen bacterial isolates or the control bacterium, B. subtilis, demonstrated any growth over seven days on the ISTOR medium. The seven fungal isolates and a control fungus, Jeniculosporium, all demonstrated growth in the ISTOR medium.

TABLE VIII
ENVIRONMENTAL TESTING WITH ISTOR

| Bacterial Isolate | Growth | Fungal Isolate | Growth |
|---|---|---|---|
| #1 | − | #1 | + |
| #2 | − | #2 | + |
| #3 | − | #3 | + |
| #4 | − | #4 | + |
| #5 | − | #5 | + |
| #6 | − | #6 | + |
| #7 | − | #7 | + |
| #8 | − | #8 | + |
| #9 | − | Con: Jeniculospsorium | + |
| #10 | − | | |
| #11 | − | | |
| #12 | − | | |
| #13 | − | | |
| Con: B. subtilis | − | | |

Other common fungi that exhibited positive growth on ISTOR medium include Penicillium sp., Aspergillus sp., and Trametes versicolor. Additional fungi that were found to grow in ISTOR medium are Cladosporium sp., Aureobasidium sp. and Phanerochaete chrysosporium.

It is clear from this testing that the fungal assay of the present invention is capable of detecting fungi from an environmental sample, i.e., for a sample containing a mixture of microorganisms, unknown species, and unknown numbers of fungi.

Once the bacterial growth has been inhibited, the fungi can be detected by any conventional means. Among the detection methods are those disclosed in Haugland et al., U.S. Pat. No. 6,387,652; Miller et al., U.S. Pat. No. 6,372,226; Laine et al., U.S. Pat. No. 6,090,573; Mayer et al., U.S. Pat. No. 5,789,191; Godsey et al., U.S. Pat. No. 5,888,760; Dorn et al., U.S. Pat. No. 4,144,133; and Bar-or et al., U.S. Pat. No. 5,098,830, the entire contents of which are hereby incorporated by reference.

The foregoing description of the specific embodiments of the present invention will so fully reveal the general nature of the invention that others can, by applying current knowledge, readily modify and/or adapt for various application such specific embodiments without undue experimentation and without departing from the generic concept. Therefore, such adaptations and modifications should and are intended to be comprehended within the meaning and range of equivalents of the disclosed embodiments.

It is to be understood that the phraseology or terminology employed herein is for the purpose of description and not of limitation. The means and materials for carrying out disclosed functions may take a variety of alternative forms without departing from the invention. Thus, the expressions “means to . . . ” and “means for . . . ” as may be found the specification above, and/or in the claims below, followed by a functional statement, are intended to define and cover whatever structural, physical, chemical, or electrical element or structures which may now or in the future exist for carrying out the recited function, whether or not precisely equivalent to the embodiment or embodiments disclosed in the specification above, and it is intended that such expressions be given their broadest interpretation.

1. A method for inhibiting bacterial growth in a culture medium for bacteria and fungi comprising adding to the culture medium an effective amount of a compound that inhibits a pathway by which bacteria synthesize lysine to inhibit bacterial growth.

2. The method according to claim 1 wherein the compound that inhibits a pathway by which bacteria synthesize lysine is selected from the group consisting of nicotinic acid, analogues of nicotinic acid, analogues of dihydropicolinic acid, analogues of diaminopimelic acid, and mixtures thereof.

3. The method according to claim 2 wherein the compound that inhibits a pathway by which bacteria synthesize lysine is nicotinic acid.

4. The method according to claim 1 wherein the culture medium is ISTOR medium.

5. The method according to claim 1 wherein the pH of the culture medium ranges from about 5.5 to about 6.5.

6. In a method for analyzing a sample for fungi wherein the sample contains bacteria and fungi, the improvement comprising adding to the culture medium an effective amount of a compound that inhibits a pathway by which bacteria synthesize lysine to inhibit bacterial growth.

7. The method according to claim 6 wherein the compound that inhibits a pathway by which bacteria synthesize lysine to inhibit bacterial growth is selected from the group consisting of nicotinic acid, analogues of nicotinic acid, analogues of dihydropicolinic acid, analogues of diaminopimelic acid, and mixtures thereof.

8. The method according to claim 7 wherein the compound that inhibits a pathway by which bacteria synthesize lysine to inhibit bacterial growth is nicotinic acid.

9. In a culture medium for assaying for fungi in the presence of bacteria in the sample, the improvement comprising adding to the culture medium an effective amount of a compound that inhibits a pathway by which bacteria synthesize lysine to inhibit bacterial growth.

10. The culture medium according to claim 9 wherein the compound that inhibits a pathway by which bacteria synthesize lysine to inhibit bacterial growth is selected from the group consisting of nicotinic acid, analogues of nicotinic acid, analogues of dihydropicolinic acid, analogues of diaminopimelic acid, and mixtures thereof.

11. The culture medium according to claim 10 wherein the compound that inhibits a pathway by which bacteria synthesize lysine to inhibit bacterial growth is nicotinic acid.
